(19) United States
(12) Patent Application Publication
(10) Pub. No.: US 2006/0057096 A1
(43) Pub. Date: Mar. 16, 2006

(54) Cosmetic composition comprising at least one cationic surfactant, at least one aminated silicone, at least one fatty alcohol, and at least one diol

(76) Inventors: Pascale Lazzeri, Levallois Perret (FR); Sandrine Decoster, Saint Gratien (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/219,763

(22) Filed: Sep. 7, 2005

Related U.S. Application Data
(60) Provisional application No. 60/612,873, filed on Sep. 27, 2004.

(30) Foreign Application Priority Data
Sep. 8, 2004 (FR) 04 51990

(51) Int. Cl. A61K 8/41

(52) U.S. Cl. 424/70.121; 424/70.28

(57) ABSTRACT

Disclosed herein is a cosmetic composition, in particular a hair composition, comprising, in a cosmetically acceptable medium, at least one quaternary ammonium cationic surfactant, at least one aminated silicone, at least one fatty alcohol, and at least one diol comprising 6 carbon atoms.

This application claims benefit of U.S. Provisional Application No. 60/612,873, filed Sep. 27, 2004, the contents of which are incorporated herein by reference. This application also claims benefit of priority under 35 U.S.C. § 119 to French Patent Application No. 04/51990, filed Sep. 8, 2004, the contents of which are also incorporated by reference.

Disclosed herein are novel cosmetic compositions, for example, hair compositions, comprising at least one cationic surfactant, at least one aminated silicone, at least one fatty alcohol, and at least one diol. Also disclosed is the use of these compositions in cosmetics, for example, in conditioners.

Conventional conditioners comprising a cationic surfactant and fatty alcohols may make it possible to obtain good cosmetic properties for the hair. However, the user may sometimes have the feeling that the hair is heavy and lank, and that it easily returns to a greasy condition, particularly if the hair is fine. Furthermore, for some hair types, the cosmetic properties of the conventional conditioner may be inadequate. In addition, the distribution of the product is sometimes difficult.

The present inventors have discovered that compositions comprising at least one cationic surfactant, at least one quaternized or non-quaternized aminated silicone, at least one fatty alcohol, and at least one diol comprising 6 carbon atoms, in certain ratios between the various constituents, may overcome the abovementioned disadvantages.

These compositions may also make it possible to render the fiber more supple, i.e., render the fiber less rough and more malleable.

The inventors have also found that, during application to fibers such as hair, the compositions may be easily distributed and easily spread from the roots to the ends, and/or may be easier to rinse out.

Finally, these compositions may confer good cosmetic properties on keratin fibers, such as the hair, for example, the hair may have improved disentangling and/or smoothness, the hair may be light and return more slowly to a greasy condition, and/or the hair may be very easily and/or lastingly shaped.

One embodiment of the present disclosure is thus a non-coloring cosmetic composition, for example, a hair composition, comprising, in a cosmetically acceptable medium, at least one cationic surfactant chosen from quaternary ammonium salts, at least one quaternized or non-quaternized aminated silicone, at least one fatty alcohol, and at least one diol comprising 6 carbon atoms, wherein the ratio of the weight concentrations of fatty alcohol/cationic surfactant is greater than or equal to 1, the ratio of the weight concentrations of cationic surfactant/silicone is greater than or equal to 0.85 and the ratio of the weight concentrations of fatty alcohol/silicone ranges from 2 to 15.

Another embodiment of the disclosure is a cosmetic haircare process comprising applying the compositions of the disclosure to the hair.

A further embodiment of the instant disclosure is a process for the cosmetic treatment of the hair and/or scalp comprising applying the compositions applying the compositions of the present disclosure to the hair and/or scalp.

Still a further embodiment of the present disclosure is a process for conditioning keratinous substances, such as the hair, comprising applying the compositions disclosed herein to the keratinous substances.

Other characteristics, aspects, subject-matters and advantages of the present disclosure will become even more clearly apparent on reading the description and examples which follow.

As used herein, the term “at least one” will be understood as meaning “one or more.”

As used herein, the term “cosmetically acceptable medium” is understood to mean a medium compatible with keratinous substances, such as the skin, hair, nails, eyelashes, eyebrows, lips, and/or any other region of the body and/or face.

As used herein, the term “non-coloring composition” is understood to mean a composition comprising essentially no oxidation dye, for example, less than 0.5% by weight, or less than 0.1% by weight with respect to the total weight of the composition, or comprising no oxidation dye.

The cosmetically acceptable medium may comprise at least one medium chosen from water, from cosmetically acceptable solvents, for example, hydrophilic solvents, and from water-solvent mixtures. These solvents may be alcohols, for example, linear or branched C₁-C₆ monoalcohols, such as ethanol, tert-butanol, n-butanol, isopropanol, and n-propanol.

The cosmetically acceptable medium may be present in the composition in an amount ranging from 30% to 99% by weight, with respect to the total weight of composition, for example, from 60% to 90% by weight.

Cationic Surfactants

Examples of quaternary ammonium salts suitable for use as cationic surfactants in the compositions disclosed herein include alkylpyridinium salts, imidazoline ammonium salts, di(quaternary ammonium) salts, and ammonium salts comprising at least one ester functional group.

Non-limiting examples of quaternary ammonium salts useful herein include:
quaternary ammonium salts of formula (I): 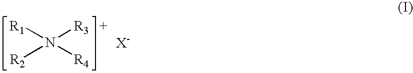
   in which:
R₁, R₂, R₃, and R₄, which may be identical or different, are chosen from linear or branched aliphatic radicals comprising from 1 to 30 carbon atoms and from aromatic radicals, such as aryl and alkylaryl radicals. The aliphatic radicals may comprise heteroatoms, such as oxygen, nitrogen, and sulphur, and/or halogen atoms. The aliphatic radicals may be chosen from, for example, alkyl radicals, such as C₁₂-C₂₂ alkyl radicals; alkoxy radicals; polyoxy(C₂-C₆)alkylene radicals; alkylamide radicals; (C₁₂-C₂₂)alkylamido(C₂-C₆)alkyl radicals; (C₁₂-C₂₂)alkyl acetate radicals; and hydroxyalkyl radicals comprising from 1 to 30 carbon atoms; and
X⁻ is an anion chosen from halides, phosphates, acetates, lactates, (C₂-C₆)alkyl sulphates, alkylsulphonates, and alkylarylsulphonates;
imidazoline quaternary ammonium salts, for example, those of formula (II): 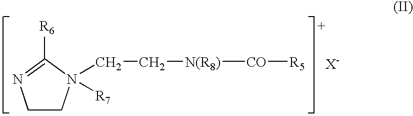
   in which:
R₅ is chosen from alkenyl and alkyl radicals comprising from 8 to 30 carbon atoms, for example, derivatives of tallow and derivatives of coconut fatty acids,
R₆ is chosen from hydrogen, C₁-C₄ alkyl radicals, alkenyl radicals comprising from 8 to 30 carbon atoms, and alkyl radicals comprising from 8 to 30 carbon atoms,
R₇ is chosen from C₁-C₄ alkyl radicals,
R₈ is chosen from hydrogen and C₁-C₄ alkyl radicals, and
X⁻ is an anion chosen from halides, phosphates, acetates, lactates, alkyl sulphates, alkylsulphonates, and alkylarylsulphonates. In one embodiment, R₅ and R₆ are chosen from mixtures of alkenyl radicals comprising from 12 to 21 carbon atoms and mixtures of alkyl radicals comprising from 12 to 21 carbon atoms, for example, derivatives of tallow fatty acids, R₇ is methyl, and R₈ is hydrogen. This embodiment may include, for example, commercial products such as Quaternium-27 (CTFA 1997) and Quaternium-83 (CTFA 1997), which are sold under the names “Rewoquat®” W 75, W90, W75PG, and W75HPG by Witco;
di(quaternary ammonium) salts of formula (III): 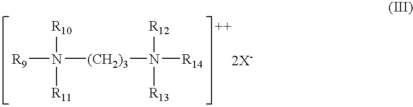
   in which:
R₉ is chosen from aliphatic radicals comprising from 16 to 30 carbon atoms,
R₁₀, R₁₁, R₁₂, R₁₃, and R₁₄, which may be identical or different, are chosen from hydrogen and alkyl radicals comprising from 1 to 4 carbon atoms, and
X⁻ is an anion chosen from halides, acetates, phosphates, nitrates, ethyl sulphates, and methyl sulphates. A non-limiting example of such di(quaternary ammonium) salts is propanetallowdiammonium dichloride.
quaternary ammonium salts comprising at least one ester functional group, such as those of formula (IV): 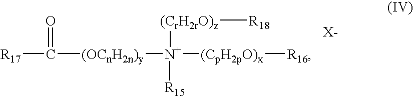
   in which:
R₁₅ is chosen from C₁-C₆ alkyl radicals, C₁-C₆ hydroxyalkyl radicals, and C₁-C₆ dihydroxyalkyl radicals;
R₁₆ is chosen from 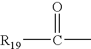
   radicals, saturated or unsaturated, linear or branched C₁-C₂₂ hydrocarbon radicals R₂₀, and hydrogen,
R₁₈ is chosen from 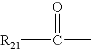
   radicals, saturated or unsaturated, linear or branched C₁-C₆ hydrocarbon radicals R₂₂, and hydrogen,
R₁₇, R₁₉, and R₂₁, which may be identical or different, are chosen from saturated or unsaturated, linear or branched C₇-C₂₁ hydrocarbon radicals;
r, n, and p, which may identical or different, are integers ranging from 2 to 6;
y is an integer ranging from 1 to 10;
x and z, which may be identical or different, are integers ranging from 0 to 10;
X⁻ is an organic or inorganic, simple or complex anion; with the proviso that the sum x+y+z ranges from 1 to 15, that when x=0, then R₁₆ denotes R₂₀, and that when z=0, then R₁₈ denotes R₂₂.

The R₁₅ alkyl radicals may be linear or branched. In one embodiment, the R₁₅ alkyl radicals are linear. For example, R₁₅ may be chosen from methyl, ethyl, hydroxyethyl, and dihydroxypropyl radicals. In another aspect, R₁₅ may be chosen from methyl and ethyl radicals.

According to one embodiment of the instant disclosure, the sum x+y+z may range from 1 to 10.

In another embodiment, when R₁₆ is an R₂₀ hydrocarbon radical, it may comprise, for example, from 12 to 22 carbon atoms, or from 1 to 3 carbon atoms.

In at least one embodiment, when R₁₈ is an R₂₂ hydrocarbon radical, it may comprise from 1 to 3 carbon atoms.

According to another embodiment of the present disclosure, R₁₇, R₁₉, and R₂₁, which may be identical or different, may be chosen from saturated or unsaturated, linear or branched C₁-C₂₁ hydrocarbon radicals, for example, saturated or unsaturated, linear or branched C₁₁-C₂₁ alkyl and alkenyl radicals.

In a further embodiment of the disclosure, x and z, which may be identical or different, are equal to 0 or 1. In another embodiment, y is equal to 1. In yet another embodiment, r, n, and p, which may be identical or different, are equal to 2 or 3. In still a further embodiment, r, n, and p are equal to 2.

The anion X⁻ may be chosen from halides, for example, chloride, bromide, and iodide, and (C₁-C₄)alkyl sulphates, such as methyl sulphate. Other suitable anions include, but are not limited to, methanesulphonate, phosphate, nitrate, tosylate, anions derived from organic acids, such as acetate or lactate, and any other anions compatible with the ammonium comprising an ester functional group. In one embodiment, the anion X⁻ is chosen from chloride and methyl sulphate.

Other suitable quaternary ammonium salts include those of formula (IV) in which:
R₁₅ is chosen from methyl and ethyl radicals,
x and y are equal to 1;
z is equal to 0 or 1;
r, n, and p are equal to 2;
R₁₆ is chosen from 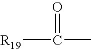
   radicals, methyl radicals, ethyl radicals, and C₁₄-C₂₂ hydrocarbon radicals, and hydrogen;
R₁₈ is chosen from 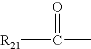
   radicals and hydrogen;
R₁₇, R₁₉, and R₂₁, which may be identical or different, are chosen from saturated or unsaturated, linear or branched C₁₃-C₁₇ hydrocarbon radicals, for example, saturated or unsaturated, linear or branched C₁₃-C₁₇ alkyl and alkenyl radicals. In one embodiment, the C₁₃-C₁₇ hydrocarbon radicals are linear.

Examples of compounds of formula (IV) include salts (for example chlorides and methyl sulphates) of diacyloxyethyldimethylammonium diacyloxyethyl(hydroxyethyl) methylammonium, monoacyloxyethyldi(hydroxyethyl)methylammonium, triacyloxyethylmethylammonium, monoacyloxyethyl(hydroxyethyl)dimethylammonium, and mixtures thereof. In one embodiment, the acyl radicals may comprise from 14 to 18 carbon atoms and may be derived from plant oils, such as palm and sunflower oils. When the compound comprises several acyl radicals, these radicals be identical or different.

These products may be obtained, for example, by direct esterification of triethanolamine, triisopropanolamine, alkyldiethanolamine or alkyldiisopropanolamine, optionally oxyalkylenated, with fatty acids or with mixtures of fatty acids of vegetable or animal origin, or by transesterification of their methyl esters. This esterification may be followed by quaternization using at least one alkylating agent chosen from alkyl halides (for example, methyl halides and ethyl halides), dialkyl sulphates (for example, dimethyl sulphate and diethyl sulphate), methyl methanesulphonate, methyl para-toluenesulphonate, ethylene chlorohydrin, and chlorohydrin.

Such compounds are, for example, sold under the names Dehyquart® by Cognis, Stepanquat® by Stepan, Noxamium® by Ceca, and Rewoquat® WE 18 by Rewo-Goldschmidt.

The composition according to the disclosure may comprise a mixture of quaternary ammonium mono-, di-, and triester salts, with a majority by weight of diester salts.

Examples of mixtures of ammonium salts that may be used include, but are not limited to, mixtures comprising 15 to 30% by weight of acyloxyethyldi(hydroxyethyl)-methylammonium methyl sulphate, 45 to 60% of diacyloxyethyl(hydroxyethyl)methyl-ammonium methyl sulphate, and 15 to 30% of triacyloxyethylmethylammonium methyl sulphate, wherein the acyl radicals may comprise from 14 to 18 carbon atoms and may be derived from palm oil which is optionally partially hydrogenated.

It is also possible to use quaternary ammonium salts comprising at least one ester functional group, such as those disclosed in U.S. Pat. Nos. 4,874,554 and 4,137,180.

In at least one embodiment of the present disclosure, the quaternary ammonium salts corresponding to formula (I) are used in the compositions disclosed herein. Mention may be made of dipalmitoylethylhydroxyethylmethylammonium salts (for example, methyl sulphates) and tetraalkylammonium salts (for example, chlorides), such as, dialkyldimethylammonium and alkyltrimethylammonium salts in which the alkyl radical comprises from 12 to 22 carbon atoms, for example, behenyltrimethylammonium, distearyldimethylammonium, cetyltrimethylammonium, and benzyldimethylstearyl-ammonium salts (for example, chlorides). In addition, mention may be made of, palmitylamidopropyltrimethylammonium salts (for example, chlorides) and stearamidopropyldimethyl(myristyl acetate)ammonium salts (for example, chloride), such as the stearamidopropyldimethyl(myristyl acetate)ammonium chloride sold under the name Ceraphyl® 70 by Van Dyk.

In at least one embodiment, the cationic surfactants used in the presently disclosed compositions are quaternary ammonium salts chosen from behenyltrimethylammonium chloride, dipalmitoylethyl(hydroxyethyl)methylammonium methyl sulphate, cetyltrimethylammonium chloride, quaternium-83, behenylamido-propyl(2,3-dihydroxypropyl)dimethylammonium chloride, and palmitylamidopropyl-trimethylammonium chloride.

The at least one cationic surfactant may be present in the composition in an amount ranging from 0.01 to 10%, for example, from 0.1 to 5%, or from 0.2 to 4%, by weight relative to the total weight of the composition.

Aminated Silicones

As used herein, the term “aminated silicone” denotes any silicone comprising at least one group chosen from primary amines, secondary amines, tertiary amines, and quaternary ammonium groups. Examples of such aminated silicones include, but are not limited to,
a) polysiloxanes named “amodimethicone” in the CTFA dictionary and corresponding to formula (IV): 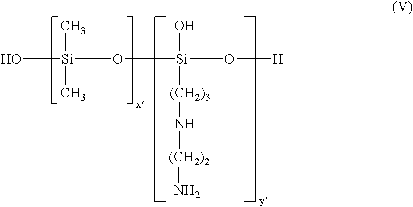
   in which:
x′ and y′ are integers dependent on the weight average molecular weight, generally having values such that the weight-average molecular weight ranges from 5,000 to 500,000;
b) aminated silicones corresponding to formula (VI):
   R′ₐG₃₋ₐ-Si(OSiG₂)ₙ-(OSiG_{b}R′_{2-b})ₘ—O-SiG₃₋ₐ-R′ₐ (VI)
   in which:
G is chosen from hydrogen, phenyl groups, OH groups, and C₁-C₈ alkyl groups, for example, a methyl group,
a is an integer ranging from 0 to 3, for example, 0,
b is equal to 0 or 1, for example, 1,
m is a number ranging from 1 to 2000, for example, from 1 to 10, and n is a number ranging from 0 to 1999, for example, from 49 to 149, wherein the sum (n+m) may range from 1 to 2000, for example, from 50 to 150,
R′ is a monovalent radical of formula —C_{q}H_{2q}L, in which q is a number ranging from 2 to 8 and L is an optionally quaternized amino group chosen from:
—NR″-Q-N′(R″)₂
—N(R″)₂
—N⁺(R″)₃A⁻
—NH⁺(R″)₂A⁻
—NH₂⁺(R″) A⁻
—N(R″)-Q-N⁺R″H₂A⁻
—NR″-Q-N⁺(R″)₂H A⁻
—NR″-Q-N⁺(R″)₃A⁻
in which R″ is chosen from hydrogen, phenyl radicals, benzyl radicals, and saturated monovalent hydrocarbon radicals, for example, alkyl radicals comprising from 1 to 20 carbon atoms; Q is chosen from linear or branched groups of formula CᵣH₂ᵣ, wherein r is an integer ranging from 2 to 6, for example, from 2 to 4, and A⁻ is chosen from halide ions, such as fluoride, chloride, bromide, and iodide.

A non-limiting example of such silicones is “trimethylsilylamodimethicone,” corresponding to formula (VII): 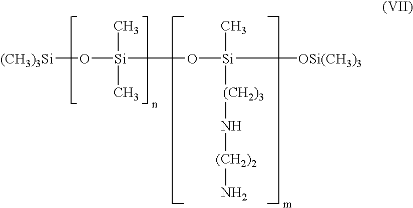
in which m is a number ranging from 1 to 2000, for example, from 1 to 10, and n is a number ranging from 0 to 1999, for example, from 49 to 149, wherein the sum (n+m) may range from 1 to 2000, for example, from 50 to 150. Such polymers are described, for example, in European Patent Application No. 0 095 238 A.
c) aminated silicones corresponding to formula (VIII): 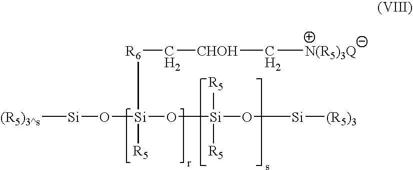
   in which:
R₅ is chosen from monovalent hydrocarbon radicals comprising from 1 to 18 carbon atoms, for example, C₁-C₁₈ alkyls, such as methyl, and C₂-C₁₈ alkenyl radicals;
R₆ is chosen from divalent hydrocarbon radicals, for example, C₁-C₁₈ alkylene radicals, and divalent C₁-C₁₈ alkyleneoxy radicals, for example, C₁-C₈ radicals, connected to Si via an Si—C bond;
Q⁻ is an anion, for example, an anion chosen from halide ions such as chloride, and organic acid salts (for example, acetate and the like);
r is a number representing a mean statistical value ranging from 2 to 20, for example, from 2 to 8;
s is a number representing a mean statistical value ranging from 20 to 200, for example, from 20 to 50. Such aminated silicones are disclosed, for example, in U.S. Pat. No. 4,185,087.
d) the quaternary ammonium silicones of formula: 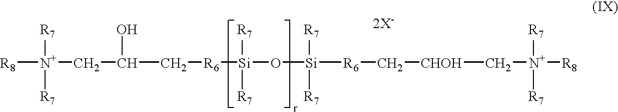
   in which:
R₇, which may be identical or different, are chosen from monovalent hydrocarbon radicals comprising from 1 to 18 carbon atoms, for example, C₁-C₁₈ alkyl radicals such as methyl radicals, C₂-C₁₈ alkenyl radicals, and rings comprising from 5 to 6 carbon atoms;
R₆ is chosen from divalent hydrocarbon radicals, for example, C₁-C₁₈ alkylene radicals, and divalent C₁-C₁₈ alkyleneoxy radicals, for example, C₁-C₈ radicals, connected to Si via an Si—C bond;
R₈, which may be identical or different, are chosen from hydrogen, monovalent hydrocarbon radicals comprising from 1 to 18 carbon atoms, for example, C₁-C₁₈ alkyl radicals and C₂-C₁₈ alkenyl radicals, and —R₆—NHCOR₇ radicals;
X⁻ is an anion, for example, an anion chosen from halide ions, such as chloride ions, and organic acid salts (for example, acetate, and the like);
r is a number representing a mean statistical value ranging from 2 to 200, for example, from 5 to 100. These silicones are described, for example, in European Patent Application No. 0 530 974 A.
e) aminated silicones of formula (X): 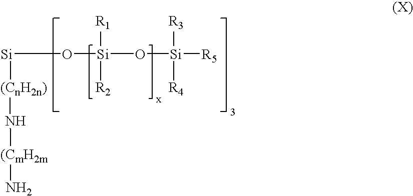
   in which:
R₁, R₂, R₃, and R₄, which may be identical or different, are chosen from C₁-C₄ alkyl radicals and phenyl groups,
R₅ is chosen from C₁-C₄ alkyl radicals and hydroxyl groups,
n is an integer ranging from 1 to 5,
m is an integer ranging from 1 to 5, and
x is a number chosen such that the amine number ranges from 0.01 to 1 meq/g.

In at least one embodiment of the present disclosure, the silicones used in the compositions disclosed herein include, but are not limited to, polysiloxanes comprising amino groups, such as amodimethicones and trimethylsilylamodimethicones (CTFA, 4^{th} edition, 1997), and silicones comprising quaternary ammonium groups.

The at least one aminated silicone may be present in the composition in an amount ranging from 0.01 to 6%, for example, from 0.1 to 3%, or from 0.4 to 2% by weight relative to the total weight of the composition.

Fatty Alcohols

The at least one fatty alcohol according to the present disclosure may be linear or branched and saturated or unsaturated, and may comprise from 8 to 40 carbon atoms. In at least one embodiment, the fatty alcohols may be oxyalkylenated or glycerolated.

The fatty alcohol may have the structure R—OH, in which R is chosen from saturated or unsaturated, linear or branched radicals comprising from 8 to 40 carbon atoms, for example, from 8 to 30 carbon atoms. In one embodiment, R may be chosen from C₁₂-C₂₄ alkyl groups and C₁₂-C₂₄ alkenyl groups. R may also be substituted with at least one hydroxyl group. In another embodiment, R is not substituted with a hydroxyl group.

Examples of suitable fatty alcohols include, but are not limited to, cetyl alcohol, stearyl alcohol, behenyl alcohol, oleyl alcohol, and mixtures thereof.

As used herein, the term “oxyalkylenated fatty alcohol” is understood to mean any pure fatty alcohol with the following structure: 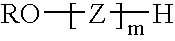
in which:
R is chosen from saturated or unsaturated, linear or branched radicals comprising from 8 to 40 carbon atoms, for example, from 8 to 30 carbon atoms,
Z is an oxyethylene radical of formula (i) and/or an oxypropylene radical chosen from oxypropylene radicals of formulas (ii)₁ and (ii)₂: 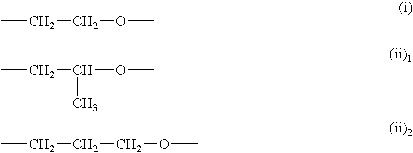
m is the number of ethylene oxide groups and/or propylene oxide groups, and may range from 1 to 250, for example, from 2 to 100.

As used herein, the term “glycerolated fatty alcohol” is understood to mean any pure fatty alcohol with the following structure: 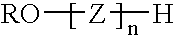
in which:
R is chosen from saturated or unsaturated, linear or branched radicals comprising from 8 to 40 carbon atoms, for example, from 8 to 30 carbon atoms,
Z is a glycerol radical of formula (iii): 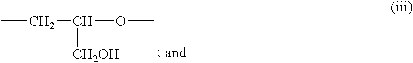
n is the number of glycerol groups, and may range from 1 to 30, for example, from 1 to 10.

In at least one embodiment of the present disclosure, the oxyalkylenated fatty alcohols used in accordance with the disclosure may be chosen from saturated or unsaturated, linear or branched fatty alcohols comprising from 10 to 20 carbon atoms and from 2 to 40 ethylene oxide groups.

Non-limiting examples of oxyalkylated fatty alcohols include the following commercial products:
MERGITAL LM2 (Cognis) [lauryl alcohol 2 EO];
IFRALAN L12 (Ifrachem) and REWOPAL 12 (Goldschmidt) [lauryl alcohol 12 EO];
EMPILAN KA 2.5/90 FL (Albright & Wilson) and MERGITAL BL309 (Cognis) [decyl alcohol 3 EO];
EMPILAN KA 5/90 FL (Albright & Wilson) and MERGITAL BL589 (Cognis) [decyl alcohol 5 EO];
BRIJ 58 (Uniquema) and SIMULSOL 58 (Seppic) [cetyl alcohol 20 EO];
EMULGIN 05 (Cognis) [oleyl/cetyl alcohol 5 EO];
MERGITAL OC30 (Cognis) [oleyl/cetyl alcohol 30 EO];
BRIJ 72 (Uniquema) [stearyl alcohol 2 EO];
BRIJ 76 (Uniquema) [stearyl alcohol 10 EO];
BRIJ 78P (Uniquema) [stearyl alcohol 20 EO];
BRIJ 700 (Uniquema) [stearyl alcohol 100 EO];
EMULGIN B1 (Cognis) [cetearyl alcohol 12 EO];
EMULGIN L (Cognis) [cetyl alcohol 9 EO and 2 PO]; and
WITCONOL APM (Goldschmidt) [myristyl alcohol 3 PO].

Examples of glycerolated fatty alcohols include, but are not limited to, lauryl alcohol comprising 4 mol of glycerol (INPCI name: polyglyceryl-4 lauryl ether), oleyl alcohol comprising 4 mol of glycerol (INPCI name: polyglyceryl-4 oleyl ether), oleyl alcohol comprising 2 mol of glycerol (INCI name: polyglyceryl-2 oleyl ether), cetearyl alcohol comprising 2 mol of glycerol, cetearyl alcohol comprising 6 mol of glycerol, oleyl/cetyl alcohol comprising 6 mol of glycerol, and octadecanol comprising 6 mol of glycerol.

The at least one fatty alcohol may also be a mixture of fatty alcohols, which means that, in a commercial product, several types of fatty alcohols can coexist in the form of a mixture.

In at least one embodiment, the fatty alcohol may be solid or pasty at a temperature of 20° C. As used herein, the term “fatty alcohol which is solid or pasty at 20° C.” is understood to mean a fatty alcohol exhibiting a viscosity, measured with a rheometer with a rate of shear of 1 s⁻¹, of greater than or equal to 1 Pa·s.

The at least one fatty alcohol of the present disclosure may be non-oxyalkylenated and/or non-glycerolated.

The at least one fatty alcohol may be present in the composition in an amount ranging from 0.1 to 15%, for example, from 0.5 to 10%, or from 2 to 8% by weight relative to the total weight of the composition.

Diols

As used herein, the term “diol comprising 6 carbon atoms” is understood to mean any hydrocarbon compound comprising 2 hydroxyl functional groups and 6 carbon atoms.

The at least one diol may be linear or branched and may comprise ether functional groups. The diol may be chosen from, for example, 1,6-hexanediol, dipropylene glycol, and mixtures thereof.

The at least one diol may be present in the composition in an amount ranging from 0.1 to 10%, for example, from 0.25 to 5%, or from 0.5 to 3% by weight relative to the total weight of the composition.

The ratio of the weight concentrations of fatty alcohol/cationic surfactant may range from 1 to 10, for example, from 1 to 5.

The ratio of the weight concentrations of cationic surfactant/silicone may range from 0.85 to 10, for example, from 0.85 to 5.

The ratio of the weight concentrations of fatty alcohol/silicone may range from 2 to 10, for example, from 2 to 5.

The composition according to the present disclosure may also comprise at least one additive chosen from fragrances, UV screening agents, nonionic surfactants, anionic surfactants, amphoteric surfactants, zwitterionic surfactants, preservatives, proteins, vitamins, provitamins, nonionic polymers, anionic polymers, cationic polymers, amphoteric polymers, and zwitterionic polymers, mineral oils, vegetable oils, and synthetic oils, non-aminated silicones, vegetable waxes, ceramides, and any other additives conventionally used in cosmetic compositions, such as antidandruff agents, agents for combating hair loss, dyes, pigments, reducing agents, and polyols, other than those disclosed above.

These additives may be present in the composition in an amount ranging from 0.001 and 20% by weight relative to the total weight of the composition. The exact amount of each additive will depend on its nature and the selected hair application, and may be readily determined by a person skilled in the art.

A person skilled in the art will take care to choose the optional additives and their amounts so that they do not harm the properties of the compositions of the present disclosure.

The compositions according to the present disclosure may be provided in a form chosen from fluids, thickened liquids, gels, creams, simple emulsions, and multiple emulsions. The compositions for the cosmetic treatment of keratinous substances in accordance with the present disclosure may also be provided in the form of a foam or mousse and may be used in rinse-out or leave-in applications. In this case, they can be packaged in an aerosol device.

The compositions of the present disclosure may be used, for example, in shampoos, conditioners, bleaching products, perming products, styling products, rinse-out care products, deep care masks, shower gels, and scalp-treatment lotions and creams, or alternatively may be deposited on wipes.

The present disclosure further relates to a method for the cosmetic treatment of the hair and/or scalp comprising applying the compositions according to the disclosure to the hair and/or scalp.

Also disclosed herein is a method for conditioning keratinous substances, such as the hair, comprising applying the compositions disclosed herein to the keratinous substances, for example, as a conditioner.

Further disclosed herein is a cosmetic treatment method comprising applying the cosmetic composition according to the disclosure to wet or dry hair, optionally followed by rinsing. In one embodiment, the composition may be applied after shampooing.

In another embodiment of the disclosure, the compositions according to the disclosure are used as conditioners for the treatment of the hair and/or scalp. In this case, the compositions may be applied to wet or dry hair, in amounts effective in treating the hair, this application optionally being followed by rinsing.

Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, unless otherwise indicated the numerical values set forth in the specific examples are reported as precisely as possible. Any numerical value, however, inherently contain certain errors necessarily resulting from the standard deviation found in their respective testing measurements.

By way of non-limiting illustration, concrete examples of certain embodiments of the present disclosure are given below. The amounts are expressed as active material, unless otherwise indicated.

### EXAMPLE 1

A conditioning composition A according to the disclosure and a comparative composition B were prepared by combining the following components:

| | Composition A | | Composition B | |
|---|---|---|---|---|
| Cetyl alcohol | 3 | g | 3 | g |
| Behenyltrimethylammonium | 1.43 | g AM | 1.43 | g AM |
| chloride | | | | |
| Dipropylene glycol | 0.57 | g AM | — | |
| Propylene glycol | — | | 0.57 | g AM |
| Myristyl/cetyl/stearyl | 1 | g | 1 | g |
| myristate/palmitate/ | | | | |
| stearate mixture | | | | |
| Polydimethylsiloxane | 0.94 | g AM | 0.94 | g AM |
| comprising an | | | | |
| aminoethyliminopropyl | | | | |
| group as a 35% aqueous | | | | |
| emulsion (DC939 | | | | |
| from Dow Corning) | | | | |
| Lanolin | 0.15 | g | 0.15 | g |
| (Lanoline USP 10/40) | | | | |
| Fragrance, Preservatives | q.s. | | q.s. | |
| pH agent q.s. | pH 4 ± 0.5 | | pH 4 ± 0.5 | |
| Water | q.s. for 100 | g | q.s. for 100 | g |

Compositions A and B were applied to the hair, followed by rinsing. Composition A was distributed more easily over the hairs from the root to the tip. Furthermore, composition A was easier to rinse out than composition B. The hair treated with composition A was also more supple under water.

It was observed that the hair treated with composition A was easier to disentangle and smoother than the hair treated with composition B.

### EXAMPLE 2

A conditioning composition A according to the disclosure and a comparative composition B were prepared by combining the following components:

| | Composition A | | Composition B | |
|---|---|---|---|---|
| Cetearyl alcohol | 5 | g | 5 | g |
| Behenyltrimethylammonium | 3.2 | g AM | 3.2 | g AM |
| chloride | | | | |
| Dipropylene glycol | 1.27 | g AM | — | |
| Isopropanol | — | | 1.27 | g AM |
| Myristyl/cetyl/stearyl | 1 | g | 1 | g |
| myristate/palmitate/ | | | | |
| stearate mixture | | | | |
| Polydimethylsiloxane | 1.05 | g AM | 1.05 | g AM |
| comprising an | | | | |
| aminoethyliminopropyl group | | | | |
| as a 35% aqueous emulsion | | | | |
| (DC939 from Dow Corning) | | | | |
| Fragrance, Preservatives | q.s. | | q.s. | |
| pH agent q.s. | pH 4 ± 0.5 | | pH 4 ± 0.5 | |
| Water | q.s. for 100 | g | q.s. for 100 | g |

Compositions A and B were applied to the hair, followed by rinsing. Composition A was distributed more easily over the hairs from the root to the tip. Furthermore, composition A was easier to rinse out than composition B. The hair treated with composition A was also more supple under water.

It was observed that the hair treated with composition A was easier to disentangle and smoother than the hair treated with composition B.

### EXAMPLE 3

A conditioning composition A according to the disclosure and a comparative composition B were prepared by combining the following components:

| | Composition A | | Composition B | |
|---|---|---|---|---|
| Cetearyl alcohol | 7 | g | 7 | g |
| Behenyltrimethylammonium | 5 | g AM | 5 | g AM |
| chloride | | | | |
| Dipropylene glycol | 2 | g AM | — | |
| Isopropanol | — | | 2 | g AM |
| Myristyl/cetyl/stearyl | 1.5 | g | 1.5 | g |
| myristate/palmitate/ | | | | |
| stearate mixture | | | | |
| Polydimethylsiloxane | 1.75 | g AM | 1.75 | g AM |
| comprising an | | | | |
| aminoethyliminopropyl group | | | | |
| as a 35% aqueous emulsion | | | | |
| (DC939 from Dow Corning) | | | | |
| Glycerol | 3 | g | 3 | g |
| Fragrance, Preservatives | q.s. | | q.s. | |
| pH agent q.s. | pH 4.5 ± 0.5 | | pH 4.5 ± 0.5 | |
| Water | q.s. for 100 | g | q.s. for 100 | g |

Compositions A and B were applied to the hair, followed by rinsing. Composition A was easier to rinse out than composition B.

It was observed that the hair treated with composition A was easier to disentangle and smoother than the hair treated with composition B.

### EXAMPLE 4

A conditioning composition A according to the disclosure and a comparative composition B were prepared by combining the following components:

| | Composition A | | Composition B | |
|---|---|---|---|---|
| Cetearyl alcohol | 7 | g | 7 | g |
| Behenyltrimethylammonium | 5 | g AM | 5 | g AM |
| chloride | | | | |
| 1,6-Hexanediol | 1.67 | g AM | — | |
| Isopropanol | — | | 1.67 | g AM |
| Myristyl/cetyl/stearyl | 1.5 | g | 1.5 | g |
| myristate/palmitate/ | | | | |
| stearate mixture | | | | |
| Polydimethylsiloxane | 1.75 | g AM | 1.75 | g AM |
| comprising an | | | | |
| aminoethyliminobutyl group | | | | |
| as a 58% aqueous emulsion | | | | |
| (DC2-8299 from Dow | | | | |
| Corning) | | | | |
| Glycerol | 3 | g | 3 | g |
| Fragrance, Preservatives | q.s. | | q.s. | |
| pH agent q.s. | pH 4.5 ± 0.5 | | pH 4.5 ± 0.5 | |
| Water | q.s. for 100 | g | q.s. for 100 | g |

Compositions A and B were applied to the hair, followed by rinsing. Composition A was easier to rinse out than composition B. The hair treated with composition A was more supple under water.

It was observed that the hair treated with composition A was easier to disentangle and smoother than the hair treated with composition B.

What is claimed is:

1. A non-coloring cosmetic composition comprising, in a cosmetically acceptable medium,
at least one cationic surfactant chosen from quaternary ammonium salts,
at least one laminated silicone,
at least one fatty alcohol, and
at least one diol comprising 6 carbon atoms,
wherein
the ratio of the weight concentrations of fatty alcohol/cationic surfactant is greater than or equal to 1,
the ratio of the weight concentrations of cationic surfactant/silicone is greater than or equal to 0.85, and
the ratio of the weight concentrations of fatty alcohol/silicone ranges from 2 to 15.

2. The composition of claim 1, wherein the quaternary ammonium salts are chosen from:
quaternary ammonium salts of formula (I): 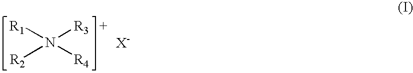
in which:
R₁, R₂, R₃, and R₄, which may be identical or different, are chosen from linear or branched aliphatic radicals comprising from 1 to 30 carbon atoms and from aromatic radicals;
X⁻ is an anion chosen from halides, phosphates, acetates, lactates, (C₂-C₆)alkyl sulphates, alkylsulphonates, and alkylarylsulphonates;
imidazoline quaternary ammonium salts of formula (II): 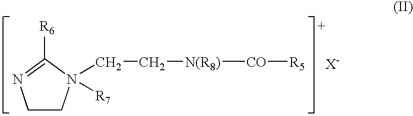
in which:
R₅ is chosen from alkenyl and alkyl radicals comprising from 8 to 30 carbon atoms,
R₆ is chosen from hydrogen, C₁-C₄ alkyl radicals, alkenyl radicals comprising from 8 to 30 carbon atoms, and alkyl radicals comprising from 8 to 30 carbon atoms,
R₇ is chosen from C₁-C₄ alkyl radicals,
R₈ is chosen from hydrogen and C₁-C₄ alkyl radicals, and
X⁻ is an anion chosen from halides, phosphates, acetates, lactates, alkyl sulphates, alkylsulphonates, and alkylarylsulphonates;
di(quaternary ammonium) salts of formula (III): 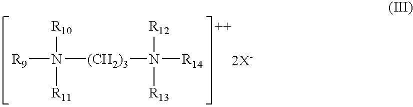
in which:
R₉ is chosen from aliphatic radicals comprising from 16 to 30 carbon atoms,
R₁₀, R₁₁, R₁₂, R₁₃, and R₁₄, which may be identical or different, are chosen from hydrogen and alkyl radicals comprising from 1 to 4 carbon atoms, and
X⁻ is an anion chosen from halides, acetates, phosphates, nitrates, ethyl sulphates, and methyl sulphates;
quaternary ammonium salts comprising at least one ester functional group of formula (IV): 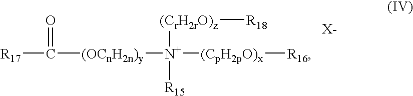
in which:
R₁₅ is chosen from C₁-C₆ alkyl radicals, C₁-C₆ hydroxyalkyl radicals, and C₁-C₆ dihydroxyalkyl radicals;
R₁₆ is chosen from 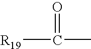
radicals, saturated or unsaturated, linear or branched C₁-C₂₂ hydrocarbon radicals R₂₀, and hydrogen,
R₁₈ is chosen from 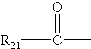
radicals, saturated or unsaturated, linear or branched C₁-C₆ hydrocarbon radicals R₂₂, and hydrogen,
R₁₇, R₁₉, and R₂₁, which may be identical or different, are chosen from saturated or unsaturated, linear or branched C₇-C₂₁ hydrocarbon radicals;
r, n, and p, which may be identical or different, are integers ranging from 2 to 6;
y is an integer ranging from 1 to 10;
x and z, which may be identical or different, are integers ranging from 0 to 10; and
X⁻ is an organic or inorganic, simple or complex anion;
with the proviso that the sum x+y+z ranges from 1 to 15, that when x has a value of 0, then R₁₆ denotes R₂₀, and that when z has a value of 0, then R₁₈ denotes R₂₂.

3. The composition of claim 2, wherein the compound of formula (IV) is chosen from diacyloxyethyldimethylammonium salts, diacyloxyethyl(hydroxyethyl)methylammonium salts, monoacyloxyethyldi(hydroxyethyl)methylammonium salts, triacyloxyethyl-methylammonium salts, monoacyloxyethyl(hydroxyethyl)dimethylammonium salts, and mixtures thereof.

4. The composition of claim 2, wherein the surfactant of formula (I) is chosen from dipalmitoylethylhydroxyethylmethylammonium salts, behenyltrimethylammonium, distearyldimethylammonium salts, cetyltrimethylammonium salts, benzyldimethylstearyl-ammonium salts, palmitylamidopropyltrimethylammonium salts, and stearamidopropyl-dimethyl(myristyl acetate)ammonium salts.

5. The composition of claim 1, wherein the at least one cationic surfactant is chosen from behenyltrimethylammonium chloride, cetyltrimethylammonium chloride, quaternium-83, behenylamidopropyl(2,3-dihydroxypropyl)dimethylammonium chloride, and palmitylamidopropyltrimethylammonium chloride.

6. The composition of claim 1, wherein the at least one cationic surfactant is present in the composition in an amount ranging from 0.01 to 10% by weight relative to the total weight of the composition.

7. The composition of claim 6, wherein the at least one cationic surfactant is present in an amount ranging from 0.1 to 5% by weight relative to the total weight of the composition.

8. The composition of claim 7, wherein the at least one cationic surfactant is present in an amount ranging from 0.2 to 4% by weight relative to the total weight of the composition.

9. The composition of claim 1, wherein the at least one aminated silicone is chosen from:
a) polysiloxanes corresponding to formula (V): 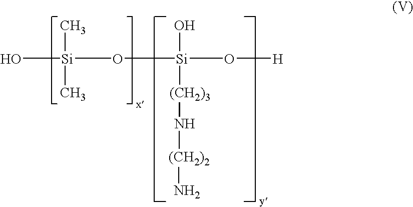
in which
x′ and y′ are integers having values such that the weight-average molecular weight ranges from 5,000 to 500,000;
b) aminated silicones corresponding to formula (VI):
R′ₐG₃₋ₐ-Si(OSiG₂)ₙ-(OsiG_{b}R_{2-b})ₘ—O-SiG₃₋ₐ-R′a (VI)
in which:
G is chosen from hydrogen, phenyl groups, OH goups, and C₁-C₈ alkyl groups,
a is an integer ranging from 0 to 3,
b is equal to 0 or 1,
m is a number ranging from 1 to 2,000 and n is a number ranging from 0 to 1999, with the proviso that the sum (n+m) has a value ranging from 1 to 2000, and
R′ is chosen from monovalent radicals of formula —C_{q}H_{2q}L in which q is a number ranging from 2 to 8 and L is an optionally quaternized amino group chosen from:
—N R″-Q-N′(R″)₂;
—N(R″)₂;
—N⁺(R″)₃ A⁻;
—NH⁺(R″)₂ A⁻;
—NH₂⁺(R″) A⁻;
—N(R″)-Q-N⁺R″H₂ A⁻;
—NR″-Q-N⁺(R″)₂H A⁻; and
—NR″-Q-N⁺(R″)₃ A⁻;
in which:
R″ is chosen from hydrogen, phenyl radicals, benzyl radicals, and saturated monovalent hydrocarbon radicals; and
Q is chosen from linear or branched groups of formula CᵣH₂ᵣ, wherein r is an integer ranging from 2 to 6 and A⁻ is chosen from halide ions,
c) aminated silicones of formula (VIII): 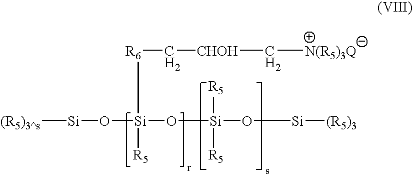
in which:
R₅ is chosen from monovalent hydrocarbon radicals comprising from 1 to 18 carbon atoms;
R₆ is chosen from divalent hydrocarbon radicals and divalent C₁-C₁₈ alkyleneoxy radicals;
Q⁻ is an anion chosen from halides and organic acid salts;
r is a number representing a mean statistical value ranging from 2 to 20; and
s is a number representing a mean statistical value ranging from 20 to 200,
d) quaternary ammonium silicones of formula (IX): 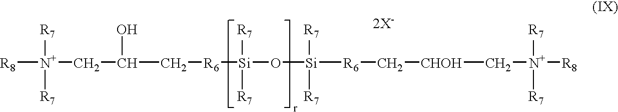
in which:
R₇, which may be identical or different, are chosen from monovalent hydrocarbon radicals comprising from 1 to 18 carbon atoms;
R₆ is chosen from divalent hydrocarbon radicals and divalent C₁-C₁₈ alkyleneoxy radicals;
R₈, which may be identical or different, are chosen from hydrogen, monovalent hydrocarbon radicals comprising from 1 to 18 carbon atoms, and —R₆—NHCOR₇ radicals;
X⁻ is an anion chosen from halides and organic acid salts; and
r is a number representing a mean statistical value ranging from 2 to 200;
e) aminated silicones of formula (X): 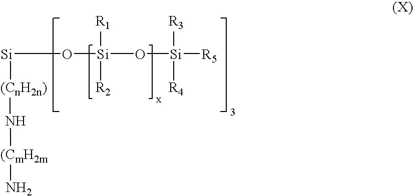
in which:
R₁, R₂, R₃, and R₄, which may be identical or different, are chosen from C₁-C₄ alkyl radicals and phenyl groups,
R₅ is chosen from C₁-C₄ alkyl radicals and hydroxyl groups,
n is an integer ranging from 1 to 5,
m is an integer ranging from 1 to 5, and
x is a number chosen such that the amine number ranges from 0.01 to 1 meq/g.

10. The composition of claim 9, wherein in formula (VI), the sum n+m ranges from 50 to 150, n ranges from 49 to 149, and m ranges from 1 to 10.

11. The composition of claim 9, wherein in formula (VIII), R₅ is chosen from C₁-C₁₈ alkyl radicals and C₂-C₁₈ alkenyl radicals.

12. The composition of claim 9, wherein in formula (VIII), R₆ is chosen from C₁-C₁₈ alkylene radicals.

13. The composition of claim 9, wherein in formula (VIII), r is a mean statistical value ranging from 2 to 8; and s is a mean statistical value ranging from 20 to 50.

14. The composition of claim 9, wherein the aminated silicone of formula (VI) is trimethylsilylamodimethicone, corresponding to formula (VII): 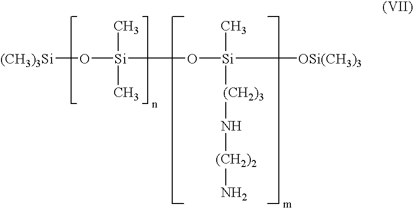
in which n is an integer ranging from 1 to 5, and m is an integer ranging from 1 to 5.

15. The composition of claim 9, wherein in formula (IX), R₇ is chosen from C₁-C₁₈ alkyl radicals, C₂-C₁₈ alkenyl radicals, and rings comprising from 5 to 6 carbon atoms.

16. The composition of claim 9, wherein in formula (IX), R₆ is chosen from C₁-C₁₈ alkylene radicals.

17. The composition of claim 9, wherein in formula (IX), R₈, is chosen from C₁-C₁₈ alkyl radicals and C₂-C₁₈ alkenyl radicals.

18. The composition of claim 9, wherein in formula (IX), r is a mean statistical value ranging from 5 to 100.

19. The composition of claim 1, wherein the at least one aminated silicone is chosen from amodimethicones and trimethylsilylamodimethicones, or silicones comprising quaternary ammonium groups.

20. The composition of claim 1, wherein the at least one aminated silicone is present in the composition in amount ranging from 0.01 to 6% by weight relative to the total weight of the composition.

21. The composition of claim 20, wherein the at least one aminated silicone is present in an amount ranging from 0.1 to 3% by weight relative to the total weight of the composition.

22. The composition of claim 21, wherein the at least one aminated silicone is present in an amount ranging from 0.4 to 2% by weight relative to the total weight of the composition.

23. The composition of claim 1, wherein the fatty alcohol is linear or branched and saturated or unsaturated.

24. The composition of claim 23, wherein the at least one fatty alcohol has the structure R—OH, in which R is chosen from saturated or unsaturated, linear or branched radicals comprising from 8 to 40 carbon atoms.

25. The composition of claim 24, wherein R is chosen from saturated or unsaturated, linear or brached radicals comprising from 8 to 30 carbon atoms.

26. The composition of claim 24, wherein the at least one fatty alcohol is chosen from cetyl alcohol, stearyl alcohol, oleyl alcohol, and mixtures thereof.

27. The composition of claim 1, wherein the fatty alcohol is solid or pasty at ambient temperature.

28. The composition of claim 1, wherein the at least one fatty alcohol is present in the composition in an amount ranging from 0.1 to 15% by weight relative to the total weight of the composition.

29. The composition of claim 28, wherein the at least one fatty alcohol is present in an amount ranging from 0.5 to 10% by weight relative to the total weight of the composition.

30. The composition of claim 29, wherein the at least one fatty alcohol is present in an amount ranging from 2 to 8% by weight relative to the total weight of the composition.

31. The composition of claim 1, wherein the at least one diol is chosen from 1,6-hexanediol, dipropylene glycol, and mixtures thereof.

32. The composition of claim 1, wherein the at least one diol is present in the composition in an amount ranging from 0.1 to 10% by weight relative to the total weight of the composition.

33. The composition of claim 32, wherein the at least one diol is present in an amount ranging from 0.25 to 5% by weight relative to the total weight of the composition.

34. The composition of claim 33, wherein the at least one diol is present in an amount ranging from 0.5 to 3% by weight relative to the total weight of the composition.

35. The composition of claim 1, wherein the ratio of the weight concentrations of fatty alcohol/cationic surfactant ranges from 1 to 10.

36. The composition of claim 35, wherein the ratio of the weight concentrations of fatty alcohol/cationic surfactant ranges from 1 to 5.

37. The composition of claim 1, wherein the ratio of the weight concentrations of cationic surfactant/silicone ranges from 0.85 to 10.

38. The composition of claim 37, wherein the ratio of the weight concentrations of cationic surfactant/silicone ranges from 0.85 to 5.

39. The composition of claim 1, wherein the ratio of the weight concentrations of fatty alcohol/silicone ranges from 2 to 5.

40. The composition of claim 1, wherein the cosmetically acceptable medium is chosen from water, cosmetically acceptable solvents, and water-solvent mixtures.

41. The composition of claim 40, wherein the cosmetically acceptable solvent is chosen from ethanol and isopropanol.

42. The composition of claim 1, further comprising at least one additive chosen from fragrances, UV screening agents, nonionic surfactants, anionic surfactants, amphoteric surfactants, and zwitterionic surfactants, preservatives, proteins, vitamins, provitamine, nonionic polymers, anionic polymers, cationic polymers, amphoteric polymers, and zwitterionic polymers, mineral oils, vegetable oils, and synthetic oils, antidandruff agents, agents for combating hair loss, dyes, pigments, reducing agents, non-aminated silicones, vegetable waxes, and ceramides.

43. A method for the cosmetic treatment or care of the hair and/or scalp comprising applying a non-coloring cosmetic composition to the hair and/or scalp, wherein the cosmetic composition comprises, in a cosmetically acceptable medium,
at least one cationic surfactant chosen from quaternary ammonium salts,
at least one aminated silicone,
at least one fatty alcohol, and
at least one diol comprising 6 carbon atoms,
wherein
the ratio of the weight concentrations of fatty alcohol/cationic surfactant is greater than or equal to 1,
the ratio of the weight concentrations of cationic surfactant/silicone is greater than or equal to 0.85, and
the ratio of the weight concentrations of fatty alcohol/silicone ranges from 2 to 15.

44. The method of claim 43, wherein the composition is applied to the hair and/or scalp after shampooing.

45. A method for the conditioning of keratin fibers comprising applying a non-coloring cosmetic composition to said fibers, wherein the cosmetic composition comprises, in a cosmetically acceptable medium,
at least one cationic surfactant chosen from quaternary ammonium salts,
at least one aminated silicone,
at least one fatty alcohol, and
at least one diol comprising 6 carbon atoms,
wherein
the ratio of the weight concentrations of fatty alcohol/cationic surfactant is greater than or equal to 1,
the ratio of the weight concentrations of cationic surfactant/silicone is greater than or equal to 0.85, and
the ratio of the weight concentrations of fatty alcohol/silicone ranges from 2 to 15.
